(19) United States
(12) Patent Application Publication
(10) Pub. No.: US 2024/0318151 A1
(43) Pub. Date: Sep. 26, 2024

(54) Modified Luciferases

(71) Applicant: Qatar Foundation for Education, Science and Community Development, Doha (QA)

(72) Inventor: Kabir Hassan Biswas, Doha (QA)

(21) Appl. No.: 18/609,655

(22) Filed: Mar. 19, 2024

Related U.S. Application Data
(60) Provisional application No. 63/453,368, filed on Mar. 20, 2023.

(51) Int. Cl. C12N 9/02

(52) U.S. Cl. CPC C12N 9/0069; C07K 2319/60; C12Y 113/12005

(57) ABSTRACT

The instant disclosure relates to methods for luciferase modification.

### CROSS REFERENCE TO RELATED APPLICATIONS

This application claims the benefit of U.S. Provisional Application No. 63/453,368 filed Mar. 20, 2023, which is incorporated herein by reference in its entirety.

### SEQUENCE LISTING

This application contains a sequence listing having the filename 5600234-01112_Sequence_Listing.xml, which is 8 KB in size, and was created on Mar. 9, 2024. The entire content of this sequence listing is incorporated herein by reference.

### BACKGROUND

In basic biology diagnostic techniques and testing techniques, luciferases are used as a reporter protein for detecting a target protein. As a reporter protein, luciferases as well as fluorescent proteins, fluorescent dyes, quantum dot, peroxidase, and the like are widely used. Fluorescent proteins, fluorescent dyes, and quantum dot have high fluorescence intensity but require excitation light, so they have drawbacks including the following:
a. they are phototoxic to cells;
b. the excitation light spectrum overlaps the fluorescence spectrum and therefore the signal-background ratio tends to be low, rendering them unsuitable for trace detection; and
c. the detector needs to be equipped with a built-in excitation light irradiator and a built-in spectral filter.

Luciferase does not require excitation light, and therefore it has none of the above-described drawbacks. Moreover, detection with luciferase is more suitable for trace detection than colorimetric methods which employ peroxidase and the like.

Luciferases that have been reported so far include wild-type firefly-derived luciferase (FLuc), NanoLuc, TurboLuc, luciferase derived from copepod (Gaussia princeps) (GLuc), luciferase derived from sea pansy (Renilla reniformis), and luciferase derived from copepod (Metridia longa) (MLuc). Japanese Patent Laying-Open No. 2014-100137 (PTL 1) and International Patent Laying-Open No. WO 2017/057752 (PTL 2) disclose an artificial luciferase (Aluc) engineered by selecting frequent amino acids from the amino acid sequence of a copepod-derived luciferase.

Recently, a miniaturized variant of an artificial luciferase (ALuc), named picALuc, with a molecular weight of 13 kDa and thus, the smallest luciferase, was reported. While picALuc was found to be as active as the ALuc, questions remained on the structural organization and residue-residue interactions in the protein. There is a need for small sized luciferases that do not cross-react with targets, have high expression levels, and have bright emission for enhanced detection sensitivity.

### SUMMARY

Provided herein are brighter picALuc that were identified by combining structural modeling, molecular dynamics (MD) simulations and mutational analysis. The picALuc enzymes described herein have increased enzymatic activity leading to more photons of light generated for the same amount of enzyme and substrate.

### BRIEF DESCRIPTION OF DRAWINGS

FIG. 1A shows GLuc structure highlighting secondary structural elements (α-helices), disulfide bridges and the equivalent N-terminal region deleted in picALuc.

FIG. 1B shows picALuc structural model generated using available GLuc structure (PDB:7D2O), disulfide bridges, secondary structural elements (α-helices), and N and C termini.

FIG. 1C shows aligned GLuc and modeled picALuc structures.

FIG. 1D illustrates surface representation of the modeled picALuc structure.

FIG. 2A shows surface representation of picALuc at the indicated period over the course of 1 microsecond (μs) Gaussian accelerated molecular dynamics (GaMD) simulation showing rapid structural evolution of the protein.

FIG. 2B shows Cα atom root mean square deviation (RMSD).

FIG. 2C shows values of picALuc obtained from GaMD simulation.

FIG. 2D shows a heat map with pairwise RMSD of Cα atom. RMSD values are in Å.

FIG. 3A shows the number of salt bridges formed during a 1 μs long GaMD simulation. FIG. 3A-1 inset shows the early phase of the simulation (0-25 ns).

FIG. 3B shows inter-residue distances of salt bridge forming pairs Glu10 and Lys13.

FIG. 3C shows inter-residue distances of salt bridge forming pairs Glu50 and Lys42.

FIG. 3D shows inter-residue distances of salt bridge forming pairs Asp94 and Lys56. Mean and median distances and fractional occupancies of each salt bridge are shown as insets.

FIG. 4A shows organization of mGL-picALuc protein and relative location of E10, E50 and D94 residues.

FIG. 4B shows fluorescence in cells expressing wild type (WT) or mutant mGL-picALuc proteins.

FIG. 4C shows bioluminescence in cells expressing wild type (WT) or mutant mGL-picALuc proteins. Data shown in B and C are mean±standard error of measurement (s.e.m.) from experiments performed in triplicates.

FIG. 40 shows bioluminescence spectra in cells expressing WT or mutant mGL-picALuc proteins. Bioluminescence data were fit to a Gaussian distribution.

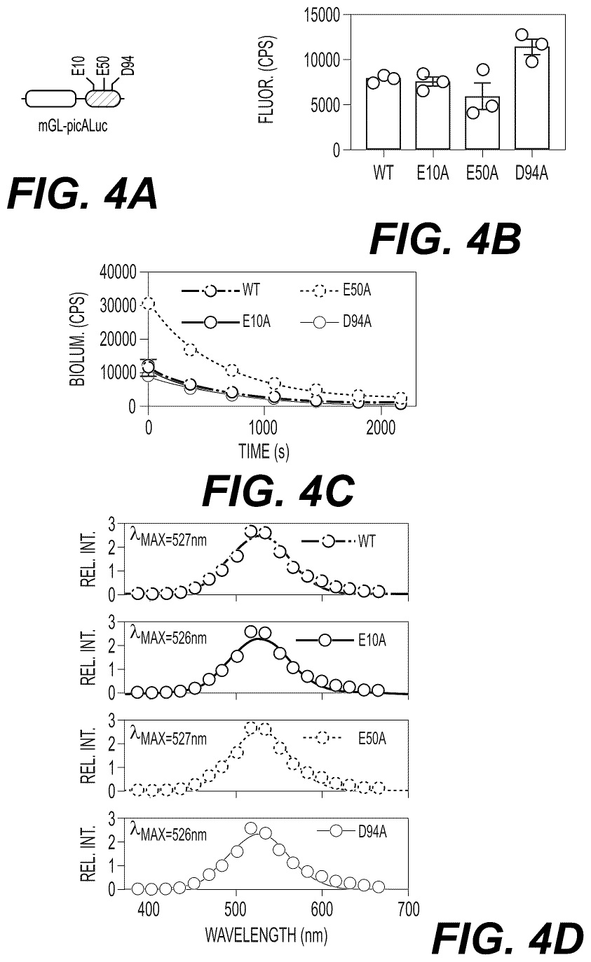

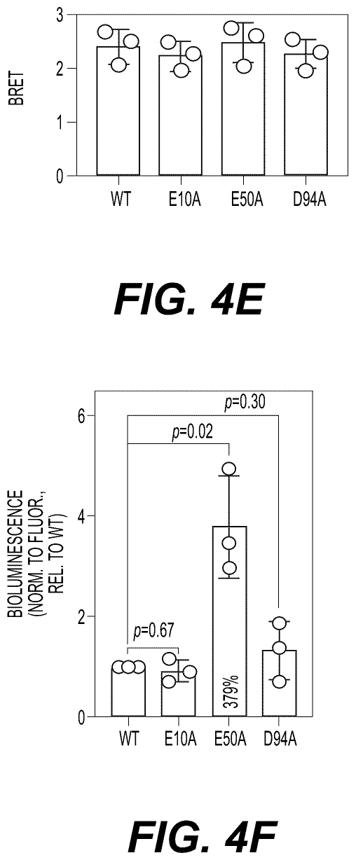

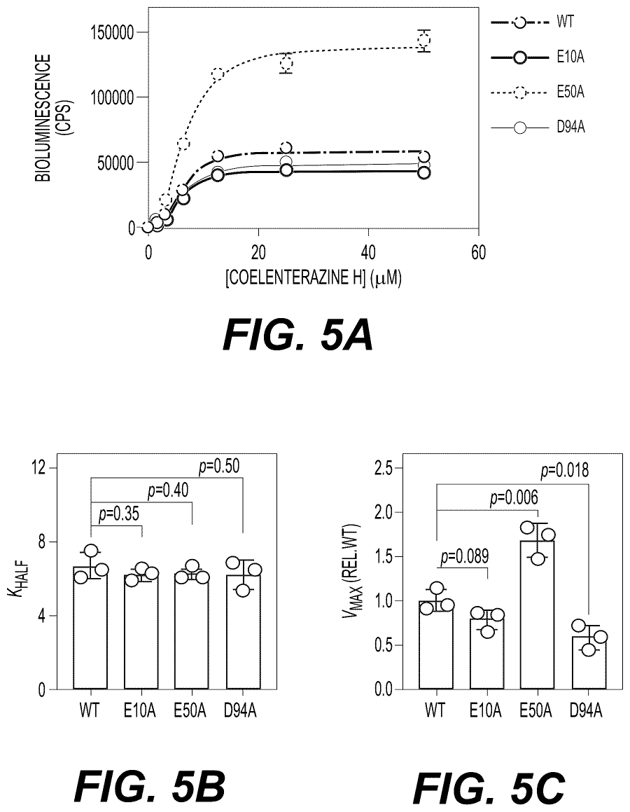

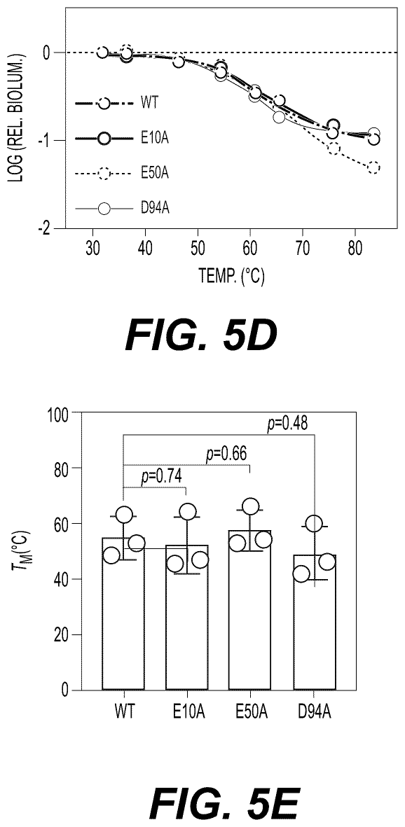

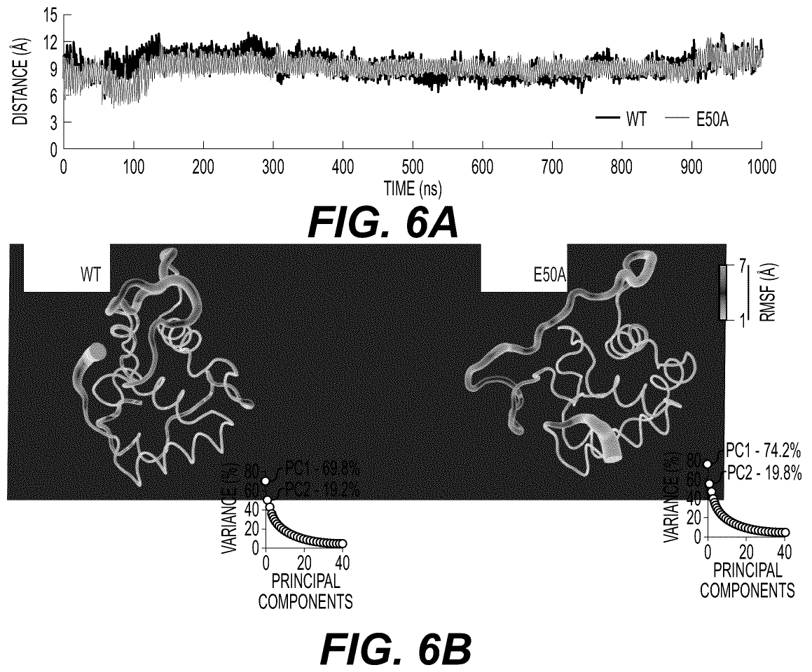

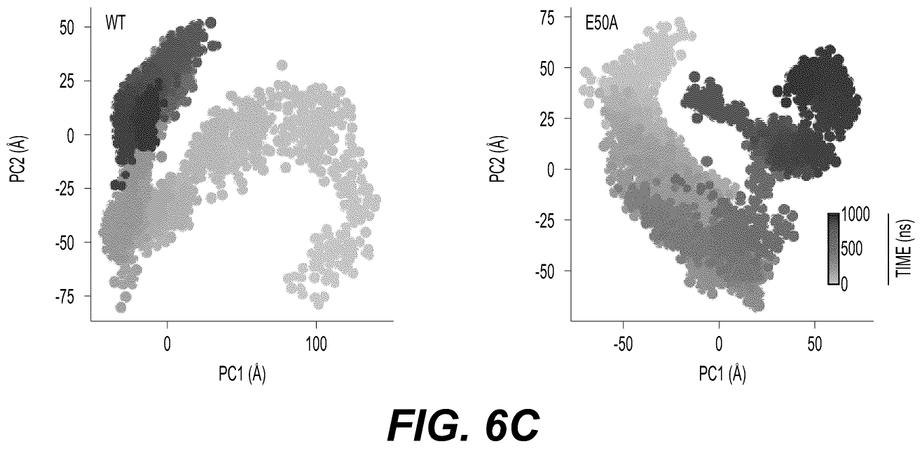

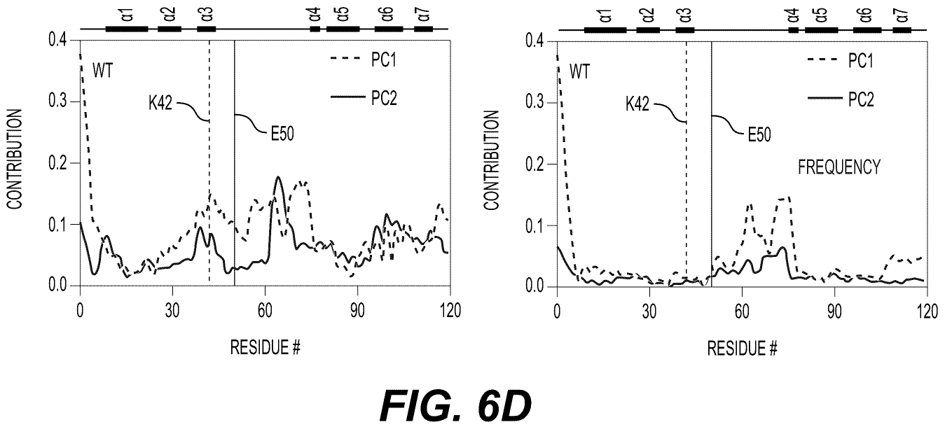

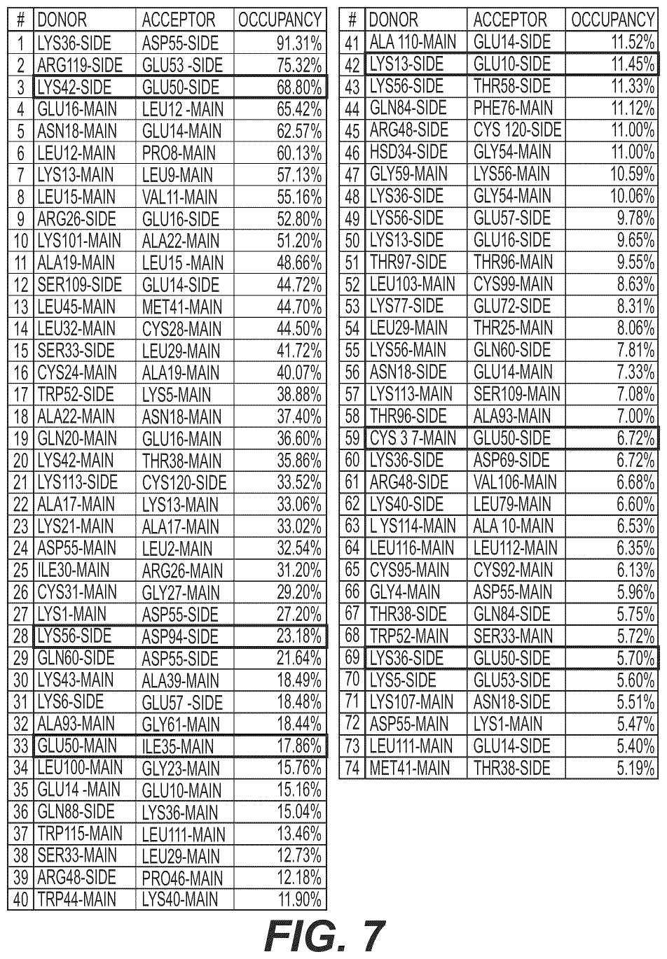

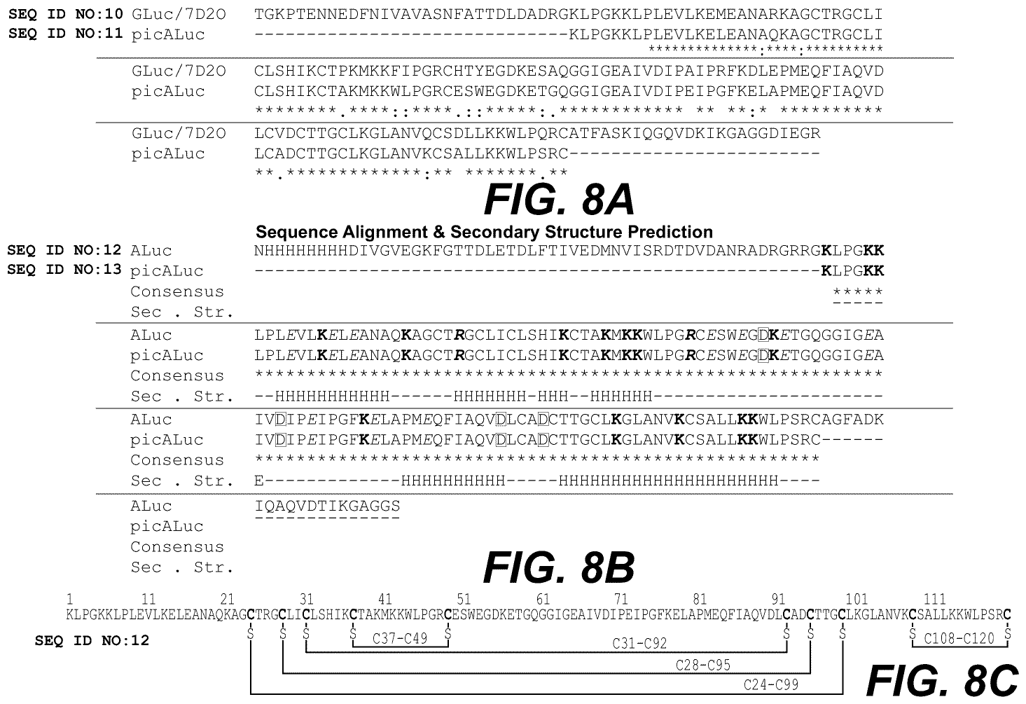

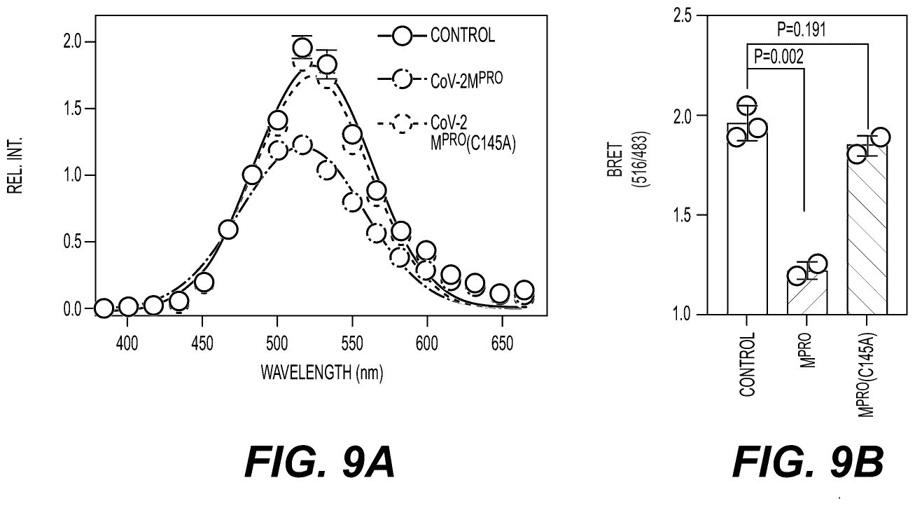

FIG. 4E shows a graph having bioluminescence resonance energy transfer (BRET) values of cells expressing WT or mutant mGL-picALuc proteins.

FIG. 4F shows graph showing bioluminescence (normalized to fluorescence values and relative to the WT or mGL-picALuc) of cells expressing WT or mutant mGL-picALuc proteins. Data shown in E and F are mean±standard deviation (sd) from three independent experiments performed in triplicates.

FIG. 5A shows a graph showing bioluminescence activity in lysates prepared from cells expressing either WT or mutant mGL-picALuc proteins under the indicated substrate (coelenterazine h) concentrations. Data shown are mean±s.e.m. of measurements from a representative experiment and fit to an allosteric sigmoidal model.

FIG. 5B shows K_{half} values of the WT or mutant mGL-picALuc proteins.

FIG. 5C shows Vₘₐₓ values of the WT or mutant mGL-picALuc proteins. Data in B and C are mean±standard deviation (sd) from three independent experiments.

FIG. 50 shows relative bioluminescence activity of either the WT or mutant mGL-picALuc protein incubated at the indicated temperatures.

FIG. 5E shows melting temperature of WT or mutant mGL-picALuc proteins obtained from Boltzmann sigmoidal model fitting of melting temperature curves. Data shown are mean±standard deviation (sd) from three independent experiments performed in triplicates.

FIG. 6A shows Cα atom distances between positions 50 and 42 (E50-K42 in the WT and A50-K42 in the E50A mutant) of picALuc over a 1 μs GaMD simulation.

FIG. 6B shows a representation of the WT (left panel) and E50A mutant (right panel) picALuc with Cα atom root mean square function (RMSF) values (Å) mapped on the conformers obtained after 1 μs GaMD simulation.

FIG. 6C shows principal components 1 and 2 (PC1 and PC2) of the WT (left panel) and E50A mutant (right panel) determined from 1 μs GaMD trajectories of each protein. Inset graphs show percentage variance against principal components obtained from principal component analysis (PCA) of the WT (left panel) and E50A mutant (right panel) picALuc.

FIG. 6D shows graphs showing contribution of individual residues to the PC1 and PC2 in the WT (left panel) and E50A mutant (right panel) picALuc simulations. Locations of residues 42 and 50 are highlighted.

FIG. 7 shows an H-bond occupancy table for H-bonds showing >5% occupancy. H-bonds formed by Glu50 side chain (rows 3, 59, 69), formed by Glu50main chain (row 33) are shown. Similarly, H-bonds formed by Glu10 side chain (row 42) and Glu10 main chain (row 35) are shown. H-bonds formed by Asp94 side chain (row 28) are shown.

FIG. 8A shows sequence alignment of GLuc (SEQ ID NO:10) and picALuc (SEQ ID NO:11).

FIG. 8B shows sequence alignment of picALuc (SEQ ID NO:12) and ALuc (SEQ ID NO:13). Positively and negatively charged residues are highlighted in blue (light blue, Lys; deep blue, Arg) and red (light red, Asp; deep red, Glu), respectively. Secondary structure prediction is shown in the lower panel.

FIG. 8C shows amino acid sequence of picALuc highlighting all disulfide bridges observed in the structural model of picALuc generated from the NMR structure of Gaussia luciferase (GLuc) [PDB: 7D2O].

FIG. 9A shows graph showing bioluminescence spectra of mGL-picALuc obtained from live cells in the absence or presence of either WT or C145A mutant SARS-CoV-2 Mₚᵣₒ.

FIG. 9B shows graph showing BRET (ratio of emissions at 516 and 483 nm) of mGL-picALuc obtained from live cells in the absence or presence of either WT or C145A mutant SARS-CoV-2 Mₚᵣₒ.

### DETAILED DESCRIPTION

Disclosed herein is the generation of a brighter version of the smallest bioluminescent (luciferase) protein that will increase the utility of an artificially created luciferase protein (see US patent #US20220259575A1 (now U.S. Ser. No. 11/827,908); incorporated by reference herein). Specifically, the brighter variant of the picALuc protein described herein can be utilized in developing biological and biomedical assays such as measuring gene expression, determining protein-protein interaction, detecting biomolecules/biomarkers, etc.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

As used herein, “WT” refers to a miniaturized variant of an artificial luciferase (ALuc), named picALuc, with a molecular weight of 13 kDa, and having an amino acid sequence corresponding to SEQ ID NO: 2.

Combining structural modeling, molecular dynamics (MD) simulations and mutational analysis, we show that the loss of a salt bridge interaction formed by Glu50 (E50) residue results in an increased enzymatic activity of picALuc. Specifically, we generated a model of picALuc using the available structure of the Gaussia luciferase (GLuc) and performed a 1 μs long Gaussian accelerated molecular dynamics (GaMD) simulation which revealed a general compaction of the protein structure as well as residue level interactions in the protein.

Given that picALuc contains a number of charged residues, we focused our attention to salt bridge interactions and decided to mutate E10, E50 and D94 that were found to form a fluctuating, stable or a new salt bridge interaction, respectively. Live cell assays showed an enhanced bioluminescence in cells expressing the E50A mutant picALuc while in vitro assays revealed an increased Vmax of the E50A mutant without affecting its thermal stability. Analysis of GaMD simulation trajectories revealed altered collective dynamics in the protein, in which residue E50 contributed substantially. We envisage that the brighter variant of picALuc reported here will find a general applicability in developing bioluminescence-based assays and the strategy developed here will pave the way for further engineering of brighter variants of picALuc.

Provided herein is a modified luciferase comprising a polypeptide having SEQ ID NO: 2 including at least one amino acid mutation, wherein the modified luciferase has a greater brightness than the polypeptide having SEQ ID NO: 2.

In some embodiments, provided herein is a modified luciferase having an amino acid sequence which differs from the sequence of a polypeptide of SEQ ID NO: 2 by including at least one amino acid mutation, wherein the modified luciferase has a greater brightness than the polypeptide of SEQ ID NO: 2.

In some embodiments, glutamine is replaced by alanine at amino acid 10, glutamine is replaced by alanine at amino acid 50, or aspartic acid is replaced by alanine at amino acid 94, or a combination thereof.

In some embodiments, glutamine is replaced by alanine at amino acid 10. In some embodiments, glutamine is replaced by alanine at amino acid 50. In some embodiments, aspartic acid is replaced by alanine at amino acid 94.

In some embodiments, provided is a modified luciferase which exhibits greater brightness than luciferase of SEQ ID NO: 2, comprising a polypeptide having an amino acid sequence which differs from SEQ ID NO: 2 in that glutamine is replaced by alanine at amino acid 50.

In some embodiments, the brightness exhibited is at least about two-fold greater than the brightness of luciferase of SEQ ID NO: 2. In some embodiments, the brightness exhibited is at about three-fold greater than the brightness of luciferase of SEQ ID NO: 2.

Provided herein is a polypeptide comprising an amino acid sequence selected from the group consisting of SEQ ID NO: 4, SEQ ID NO: 6, and SEQ ID NO: 8. In some embodiments, the amino acid sequence is SEQ ID NO: 4. In some embodiments, the amino acid sequence is SEQ ID NO: 6. In some embodiments, the amino acid sequence is SEQ ID NO: 8.

Provided is a fusion protein comprising a polypeptide comprising an amino acid sequence selected from the group consisting of SEQ ID NO: 4, SEQ ID NO: 6, and SEQ ID NO: 8.

Provided is a polynucleotide encoding a polypeptide comprising an amino acid sequence selected from the group consisting of SEQ ID NO: 4, SEQ ID NO: 6, and SEQ ID NO: 8.

In some embodiments, the polynucleotide encodes a polypeptide comprising the amino acid sequence SEQ ID NO: 4. In some embodiments, the polynucleotide encodes a polypeptide comprising the amino acid sequence SEQ ID NO: 6. In some embodiments, the polynucleotide encodes a polypeptide comprising the amino acid sequence SEQ ID NO: 8.

Provided is a vector comprising a polynucleotide encoding a polypeptide comprising an amino acid sequence selected from the group consisting of SEQ ID NO: 4, SEQ ID NO: 6, and SEQ ID NO: 8.

Provided is a host cell comprising the vector comprising a polynucleotide encoding a polypeptide comprising an amino acid sequence selected from the group consisting of SEQ ID NO: 4, SEQ ID NO: 6, and SEQ ID NO: 8.

Provided is a kit comprising a modified luciferase described above and herein.

Provided is a method of increasing the brightness of a luciferase comprising changing a salt bridge interaction of the luciferase.

Disclosed embodiments comprise bioassays and the development thereof, as well as biosensing applications.

mGL-picALuc nucleotide sequence (SEQ ID NO: 1):
ATGGGAAGTTCACATCATCATCATCATCACTCATCAGGACTGGTGCCACGGGGGTCTGAATT
CGGCATGGTGAGCAAGGGCGAGGAGCTGTTCACCGGGGTGGTGCCCATCCTGGTCGAGCTGG
ACGGCGACGTAAACGGCCACAAGTTCAGCGTCCGCGGCGAGGGCGAGGGCGATGCCACCAAC
GGCAAGCTGACCCTGAAGTTCATCTGCACCACCGGCAAGCTGCCCGTGCCCTGGCCCACCCT
CGTGACCACCTTAGGCTACGGCGTGGCCTGCTTCGCCCGCTACCCCGACCACATGAAGCAGC
ACGACTTCTTCAAGTCCGCCATGCCCGAAGGCTACGTCCAGGAGCGCACCATCTCTTTCAAG
GACGACGGTACCTACAAGACCCGCGCCGAGGTGAAGTTCGAGGGCGACACCCTGGTGAACCG
CATCGTGCTGAAGGGCATCGACTTCAAGGAGGACGGCAACATCCTGGGGCACAAGCTGGAGT
ACAACTTCAACAGCCACAAGGTCTATATCACGGCCGACAAGCAGAAGAACGGCATCAAGGCT
AACTTCAAGACCCGCCACAACGTTGAGGACGGCGGCGTGCAGCTCGCCGACCACTACCAGCA
GAACACCCCCATCGGCGACGGCCCCGTGCTGCTGCCCGACAACCACTACCTGAGCCATCAGT
CCAAACTGAGCAAAGACCCCAACGAGAAGCGCGATCACATGGTCCTGAAGGAGAGGGTGACC
GCCGCCGGGATTACACATGACATGGACGAGCTGTACAAGTACGGATCCGCGGCCGCCACCGA
GAACCTGTATGCAGTGCTCCAAAGCGGATTTCGCGGCTCTGGCAGCGCTATGAAGCTGCCCG
GCAAGAAGCTGCCCCTGGAGGTGCTGAAGGAGCTGGAGGCCAACGCCCAGAAGGCCGGCTGC
ACCAGGGGCTGCCTGATCTGCCTGAGCCACATCAAGTGCACCGCCAAGATGAAGAAGTGGCT
GCCCGGCAGGTGCGAGAGCTGGGAGGGCGACAAGGAGACCGGCCAGGGCGGCATCGGCGAGG
CCATCGTGGACATCCCCGAGATCCCCGGCTTCAAGGAGCTGGCCCCCATGGAGCAGTTCATC
GCCCAGGTGGACCTGTGCGCCGACTGCACCACCGGCTGCCTGAAGGGCCTGGCCAACGTGAA
GTGCAGCGCCCTGCTGAAGAAGTGGCTGCCCAGCAGGTGCGGTACCGACTACAAAGACCATG
ACGGTGATTATAAAGATCATGACATCGATTACAAGGATGACGATGACAAGGATATCTGA
mGL-picALuc amino acid sequence (WT) (SEQ ID NO: 2):
MGSSHHHHHHSSGLVPRGSEFGMVSKGEELFTGVVPILVELDGDVNGHKFSVRGEGEGDATN
GKLTLKFICTTGKLPVPWPTLVTTLGYGVACFARYPDHMKQHDFFKSAMPEGYVQERTISFK
DDGTYKTRAEVKFEGDTLVNRIVLKGIDFKEDGNILGHKLEYNFNSHKVYITADKOKNGIKA
NFKTRHNVEDGGVQLADHYQQNTPIGDGPVLLPDNHYLSHQSKLSKDPNEKRDHMVLKERVT
AAGITHDMDELYKYGSAAATENLYAVLOSGFRGSGSAMKLPGKKLPLEVLKELEANAQKAGC
TRGCLICLSHIKCTAKMKKWLPGRCESWEGDKETGOGGIGEAIVDIPEIPGFKELAPMEQFI
AQVDLCADCTTGCLKGLANVKCSALLKKWLPSRCGTDYKDHDGDYKDHDIDYKDDDDKDI*
In SEQ ID NO: 2 above, the residues that were
deemed suitable for mutation are underlined.
mGL-picALuc(E10A) nucleotide sequence(SEQ ID NO: 3):
ATGGGAAGTTCACATCATCATCATCATCACTCATCAGGACTGGTGCCACGGGGGTCTGAATT
CGGCATGGTGAGCAAGGGCGAGGAGCTGTTCACCGGGGTGGTGCCCATCCTGGTCGAGCTGG
ACGGCGACGTAAACGGCCACAAGTTCAGCGTCCGCGGCGAGGGCGAGGGCGATGCCACCAAC
GGCAAGCTGACCCTGAAGTTCATCTGCACCACCGGCAAGCTGCCCGTGCCCTGGCCCACCCT
CGTGACCACCTTAGGCTACGGCGTGGCCTGCTTCGCCCGCTACCCCGACCACATGAAGCAGC
ACGACTTCTTCAAGTCCGCCATGCCCGAAGGCTACGTCCAGGAGCGCACCATCTCTTTCAAG
GACGACGGTACCTACAAGACCCGCGCCGAGGTGAAGTTCGAGGGCGACACCCTGGTGAACCG
CATCGTGCTGAAGGGCATCGACTTCAAGGAGGACGGCAACATCCTGGGGCACAAGCTGGAGT
ACAACTTCAACAGCCACAAGGTCTATATCACGGCCGACAAGCAGAAGAACGGCATCAAGGCT
AACTTCAAGACCCGCCACAACGTTGAGGACGGCGGCGTGCAGCTCGCCGACCACTACCAGCA
GAACACCCCCATCGGCGACGGCCCCGTGCTGCTGCCCGACAACCACTACCTGAGCCATCAGT
CCAAACTGAGCAAAGACCCCAACGAGAAGCGCGATCACATGGTCCTGAAGGAGAGGGTGACC
GCCGCCGGGATTACACATGACATGGACGAGCTGTACAAGTACGGATCCGCGGCCGCCACCGA
GAACCTGTATGCAGTGCTCCAAAGCGGATTTCGCGGCTCTGGCAGCGCTATGAAGCTGCCCG
GCAAGAAGCTGCCCCTGGCCGTGCTGAAGGAGCTGGAGGCCAACGCCCAGAAGGCCGGCTGC
ACCAGGGGCTGCCTGATCTGCCTGAGCCACATCAAGTGCACCGCCAAGATGAAGAAGTGGCT
GCCCGGCAGGTGCGAGAGCTGGGAGGGCGACAAGGAGACCGGCCAGGGCGGCATCGGCGAGG
CCATCGTGGACATCCCCGAGATCCCCGGCTTCAAGGAGCTGGCCCCCATGGAGCAGTTCATC
GCCCAGGTGGACCTGTGCGCCGACTGCACCACCGGCTGCCTGAAGGGCCTGGCCAACGTGAA
GTGCAGCGCCCTGCTGAAGAAGTGGCTGCCCAGCAGGTGCGGTACCGACTACAAAGACCATG
ACGGTGATTATAAAGATCATGACATCGATTACAAGGATGACGATGACAAGGATATCTGA
mGL-picALuc(E10A) amino acid sequence (SEQ ID NO: 4):
MGSSHHHHHHSSGLVPRGSEFGMVSKGEELFTGVVPILVELDGDVNGHKFSVRGEGEGDATN
GKLTLKFICTTGKLPVPWPTLVTTLGYGVACFARYPDHMKQHDFFKSAMPEGYVQERTISFK
DDGTYKTRAEVKFEGDTLVNRIVLKGIDFKEDGNILGHKLEYNFNSHKVYITADKOKNGIKA
NFKTRHNVEDGGVQLADHYQQNTPIGDGPVLLPDNHYLSHQSKLSKDPNEKRDHMVLKERVT
AAGITHDMDELYKYGSAAATENLYAVLOSGFRGSGSAMKLPGKKLPLAVLKELEANAQKAGC
TRGCLICLSHIKCTAKMKKWLPGRCESWEGDKETGOGGIGEAIVDIPEIPGFKELAPMEQFI
AQVDLCADCTTGCLKGLANVKCSALLKKWLPSRCGTDYKDHDGDYKDHDIDYKDDDDKDI*
In SEQ ID NO: 4 above, the mutated residue
(compared to WT) is shown in bold.
mGL-picALuc(E50A) nucleotide sequence (SEQ ID NO: 5):
ATGGGAAGTTCACATCATCATCATCATCACTCATCAGGACTGGTGCCACGGGGGTCTGAATT
CGGCATGGTGAGCAAGGGCGAGGAGCTGTTCACCGGGGTGGTGCCCATCCTGGTCGAGCTGG
ACGGCGACGTAAACGGCCACAAGTTCAGCGTCCGCGGCGAGGGCGAGGGCGATGCCACCAAC
GGCAAGCTGACCCTGAAGTTCATCTGCACCACCGGCAAGCTGCCCGTGCCCTGGCCCACCCT
CGTGACCACCTTAGGCTACGGCGTGGCCTGCTTCGCCCGCTACCCCGACCACATGAAGCAGC
ACGACTTCTTCAAGTCCGCCATGCCCGAAGGCTACGTCCAGGAGCGCACCATCTCTTTCAAG
GACGACGGTACCTACAAGACCCGCGCCGAGGTGAAGTTCGAGGGCGACACCCTGGTGAACCG
CATCGTGCTGAAGGGCATCGACTTCAAGGAGGACGGCAACATCCTGGGGCACAAGCTGGAGT
ACAACTTCAACAGCCACAAGGTCTATATCACGGCCGACAAGCAGAAGAACGGCATCAAGGCT
AACTTCAAGACCCGCCACAACGTTGAGGACGGCGGCGTGCAGCTCGCCGACCACTACCAGCA
GAACACCCCCATCGGCGACGGCCCCGTGCTGCTGCCCGACAACCACTACCTGAGCCATCAGT
CCAAACTGAGCAAAGACCCCAACGAGAAGCGCGATCACATGGTCCTGAAGGAGAGGGTGACC
GCCGCCGGGATTACACATGACATGGACGAGCTGTACAAGTACGGATCCGCGGCCGCCACCGA
GAACCTGTATGCAGTGCTCCAAAGCGGATTTCGCGGCTCTGGCAGCGCTATGAAGCTGCCCG
GCAAGAAGCTGCCCCTGGAGGTGCTGAAGGAGCTGGAGGCCAACGCCCAGAAGGCCGGCTGC
ACCAGGGGCTGCCTGATCTGCCTGAGCCACATCAAGTGCACCGCCAAGATGAAGAAGTGGCT
GCCCGGCAGGTGCGCCAGCTGGGAGGGCGACAAGGAGACCGGCCAGGGCGGCATCGGCGAGG
CCATCGTGGACATCCCCGAGATCCCCGGCTTCAAGGAGCTGGCCCCCATGGAGCAGTTCATC
GCCCAGGTGGACCTGTGCGCCGACTGCACCACCGGCTGCCTGAAGGGCCTGGCCAACGTGAA
GTGCAGCGCCCTGCTGAAGAAGTGGCTGCCCAGCAGGTGCGGTACCGACTACAAAGACCATG
ACGGTGATTATAAAGATCATGACATCGATTACAAGGATGACGATGACAAGGATATCTGA
mGL-picALuc(E50A) amino acid sequence (SEQ ID NO: 6):
MGSSHHHHHHSSGLVPRGSEFGMVSKGEELFTGVVPILVELDGDVNGHKFSVRGEGEGDATN
GKLTLKFICTTGKLPVPWPTLVTTLGYGVACFARYPDHMKQHDFFKSAMPEGYVQERTISFK
DDGTYKTRAEVKFEGDTLVNRIVLKGIDFKEDGNILGHKLEYNENSHKVYITADKQKNGIKA
NFKTRHNVEDGGVQLADHYQQNTPIGDGPVLLPDNHYLSHQSKLSKDPNEKRDHMVLKERVT
AAGITHDMDELYKYGSAAATENLYAVLQSGFRGSGSAMKLPGKKLPLEVLKELEANAQKAGC
TRGCLICLSHIKCTAKMKKWLPGRCASWEGDKETGQGGIGEAIVDIPEIPGFKELAPMEQFI
AQVDLCADCTTGCLKGLANVKCSALLKKWLPSRCGTDYKDHDGDYKDHDIDYKDDDDKDI*
In SEQ ID NO: 6 above, the mutated residue
(compared to WT) is shown in bold.
mGL-picALuc(D94A) nucleotide sequence (SEQ ID NO: 7):
ATGGGAAGTTCACATCATCATCATCATCACTCATCAGGACTGGTGCCACGGGGGTCTGAATT
CGGCATGGTGAGCAAGGGCGAGGAGCTGTTCACCGGGGTGGTGCCCATCCTGGTCGAGCTGG
ACGGCGACGTAAACGGCCACAAGTTCAGCGTCCGCGGCGAGGGCGAGGGCGATGCCACCAAC
GGCAAGCTGACCCTGAAGTTCATCTGCACCACCGGCAAGCTGCCCGTGCCCTGGCCCACCCT
CGTGACCACCTTAGGCTACGGCGTGGCCTGCTTCGCCCGCTACCCCGACCACATGAAGCAGC
ACGACTTCTTCAAGTCCGCCATGCCCGAAGGCTACGTCCAGGAGCGCACCATCTCTTTCAAG
GACGACGGTACCTACAAGACCCGCGCCGAGGTGAAGTTCGAGGGCGACACCCTGGTGAACCG
CATCGTGCTGAAGGGCATCGACTTCAAGGAGGACGGCAACATCCTGGGGCACAAGCTGGAGT
ACAACTTCAACAGCCACAAGGTCTATATCACGGCCGACAAGCAGAAGAACGGCATCAAGGCT
AACTTCAAGACCCGCCACAACGTTGAGGACGGCGGCGTGCAGCTCGCCGACCACTACCAGCA
GAACACCCCCATCGGCGACGGCCCCGTGCTGCTGCCCGACAACCACTACCTGAGCCATCAGT
CCAAACTGAGCAAAGACCCCAACGAGAAGCGCGATCACATGGTCCTGAAGGAGAGGGTGACC
GCCGCCGGGATTACACATGACATGGACGAGCTGTACAAGTACGGATCCGCGGCCGCCACCGA
GAACCTGTATGCAGTGCTCCAAAGCGGATTTCGCGGCTCTGGCAGCGCTATGAAGCTGCCCG
GCAAGAAGCTGCCCCTGGAGGTGCTGAAGGAGCTGGAGGCCAACGCCCAGAAGGCCGGCTGC
ACCAGGGGCTGCCTGATCTGCCTGAGCCACATCAAGTGCACCGCCAAGATGAAGAAGTGGCT
GCCCGGCAGGTGCGAGAGCTGGGAGGGCGACAAGGAGACCGGCCAGGGCGGCATCGGCGAGG
CCATCGTGGACATCCCCGAGATCCCCGGCTTCAAGGAGCTGGCCCCCATGGAGCAGTTCATC
GCCCAGGTGGACCTGTGCGCCGCCTGCACCACCGGCTGCCTGAAGGGCCTGGCCAACGTGAA
GTGCAGCGCCCTGCTGAAGAAGTGGCTGCCCAGCAGGTGCGGTACCGACTACAAAGACCATG
ACGGTGATTATAAAGATCATGACATCGATTACAAGGATGACGATGACAAGGATATCTGA
mGL-picALuc(D94A) amino acid sequence (SEQ ID NO: 8):
MGSSHHHHHHSSGLVPRGSEFGMVSKGEELFTGVVPILVELDGDVNGHKESVRGEGEGDATN
GKLTLKFICTTGKLPVPWPTLVTTLGYGVACFARYPDHMKQHDFFKSAMPEGYVQERTISFK
DDGTYKTRAEVKFEGDTLVNRIVLKGIDFKEDGNILGHKLEYNFNSHKVYITADKQKNGIKA
NFKTRHNVEDGGVQLADHYQQNTPIGDGPVLLPDNHYLSHQSKLSKDPNEKRDHMVLKERVT
AAGITHDMDELYKYGSAAATENLYAVLQSGFRGSGSAMKLPGKKLPLEVLKELEANAQKAGC
TRGCLICLSHIKCTAKMKKWLPGRCESWEGDKETGQGGIGEAIVDIPEIPGFKELAPMEQFI
AQVDLCAACTTGCLKGLANVKCSALLKKWLPSRCGTDYKDHDGDYKDHDIDYKDDDDKDI*

In SEQ ID NO: 8 above, the mutated residue (compared to WT) is shown in bold.

### EXAMPLES

### Methods

Structural modeling of WT and E50A mutant picALuc: We utilize the available nuclear magnetic resonance (NMR)-derived structure of GLuc (PDB: 7D2O) (Wu N. et al. Sci Rep 10, 20069 (2020). to generate a homology-based structural model of the picALuc using the SWISS-MODEL webserver. The two proteins, picALuc and GLuc show considerable sequence similarity (92.5%; and 85% identity) with key differences in the N- and C-terminal allowing high confidence structural modelling of picALuc. Notably, picALuc lacks N-terminal α-helices 1 and 2 and some residues in the C-terminal region of ALuc. Quality of the modelled structure is assessed using the MolProbity software (version 4.4) available as a part of the SWISS-MODEL webserver with a score of 2.90 and with only two Gly residues (located in the long loop between α-helices 3 and 4) out of a total of 120 residues (1.69%; the GLuc template structure 7D2O showed 4 out 168 residues, which is equal to 2.8%) as Ramachandran plot outliers. All disulfide bridges observed in GLuc structure are maintained in the modelled picALuc structure. Structural model of the E50A mutant picALuc is generated by backbone rotamer-dependent replacement of the residue E with an A using Pymol (The PyMOL Molecular Graphics System, Version 2.0.0, Schrödinger, LLC; pymol.org; New York, NY, USA).

MD Simulations: MD simulations of the modelled WT and E50A mutant picALuc structures is performed essentially as described previously (Geethakumari A. M., et al. bioRxiv, 2022.2001.2031.478460 (2022), Ahmed W. S., et al. Front Mol Biosci 9, 846996 (2022)). Inputs files including topology and parameter files are prepared using the QwikMD plugin available in the Visual Molecular Dynamics (VMD) 1.9.3 software and simulations are performed using the Nanoscale Molecular Dynamics (NAMD) 2.13 software and CHARMM36 force field. Briefly, proteins are solvated in explicit solvent using TIP3P cubic water box containing 0.15 M NaCl for charge neutralization and periodic boundary conditions applied with a 2 fs integration time-step. Prior to production simulations, energy minimization is performed on each system for 2000 steps (4 μs). Subsequently, the systems are graduated heated from 60 K to 310 K at 1 K interval to reach the 310 K equilibrium temperature. Following thermalization, temperature is maintained at 310 K using Langevin temperature control and pressure is maintained at 1.0 atm using Nose-Hoover Langevin piston pressure control. The systems are then equilibrated for 500,000 steps (1 ns). GaMD simulations are then run using the integrated GaMD module in NAMD and its default parameters. This includes a 2 ns of conventional molecular dynamics equilibration run for collecting potential statistics that are used for calculating acceleration parameters, and another 50 ns equilibration run with the addition of boost potential and finally GaMD production runs for 1,000 ns. 400 μs preparatory runs are included before each of the equilibration steps in GaMD. All GaMD simulations are run at the “dual-boost” level with one boost potential applied to the dihedral energetic term and the other applied to the total potential energetic term with a standard deviation upper limit set to 6.0 kcal/mol. Short-range non-bonded interactions are defined at 12 Å cut-off with 10 Å switching distance, while Particle-mesh Ewald (PME) scheme is used to handle long-range electrostatic interactions at 1 Å PME grid spacing. Trajectory frames are saved every 10,000 steps (20 μs) and trajectories are analyzed using the available tools in Visual Molecular Dynamics (VMD) software and MDAnalysis package. Backbone root-mean-square deviation (RMSD), root-mean-square fluctuation (RMSF), solvent accessible surface area (SASA), radius of gyration (RoG), energy calculations, secondary structure salt bridges and H-bond occupancy are performed using VMD. Pairwise RMSD, number of H-bonds formed and S atom distances in disulfide bridges are calculated using the MDAnalysis package. Principal component analysis (PCA) is performed using the PCA module available in the MDAnalysis package. Contribution of individual residues to PC1 and 2 is calculated by projecting the principal components on Cα atoms of the proteins using modules in the MDAnalysis package followed by calculation of individual Cα atom displacement (root mean squared). Movies are prepared from 500 frames out of the 50,000 frames of the 1 μs trajectory (with a step size of 100 frames) generated using the VMD Movie Maker plugin and compiled at 20 fps using the Fiji distribution of ImageJ software. All structural images are generated using Pymol (The PyMOL Molecular Graphics System, Version 2.0.0, Schrodinger, LLC; pymol.org; New York, NY, USA).

mGL-picALuc plasmid construct generation: A codon optimized gene sequence of picALuc generated using the previously described amino acid sequence (Ohmuro-Matsuyama Y., et al. ACS Chem Biol 17, 864-872 (2022)) is synthesized and subcloned into the pcDNA3.1-mGL-NLuc plasmid using BamHI and XhoI restriction enzyme sites (GenScript, Singapore) to generate the pcDNA3.1-mGL-picALuc plasmid. Additionally, the SARS-CoV-2 M^{pro} N-terminal autocleavage sequence is included in the N-terminal and 3×FLAG-tag sequence is included at the C-terminal. Glu10Ala (E10A), Glu50Ala (E50A) and Asp94Ala (D94A) mutations are generated in the pcDNA3.1-mGL-picALuc plasmid (GenScript, Singapore).

Cell culture and transfection: HEK 293T cells are cultured in Dulbecco's Modified Eagle Medium (DMEM) supplemented with 10% fetal bovine serum, and 1% penicillin-streptomycin and grown at 37° C. in 5% CO₂. Transfections are performed using polyethyleneimine (PEI) lipid according to the manufacturers' protocol. Briefly, HEK 293T cells are seeded onto 96-well white plates before 24 h of transfection. The plasmid DNA (encoding either the WT or mutant picALucs; 125 ng/well), Opti-MEM (Invitrogen; 31985088; 25 μL/well) and PEI lipid (Sigma-Aldrich; 408727-100 mL; 0.625 μL/well) is mixed and incubated at room temperature for 30 min prior to addition to the cells. For in vitro biochemical and thermal stability assays, cells are transfected in 60 mm dishes using appropriate proportion of DNA, PEI lipid and Opti-MEM. The PEI stock solution of 2 mg/mL is prepared by diluting in sterile Milli-Q water and stored at −80° C. for subsequent usage.

Live cell assays: Live cell assays to determine fluorescence, bioluminescence and bioluminescence emission spectra are performed by transfecting HEK 293T cells with either the WT or the mutant pcDNA3.1-mGL-picALuc plasmid constructs. For SARS-CoV-2 M^{pro}-mediated cleavage of mGL-picALuc, cells transfected with pcDNA3.1-mGL-picALuc plasmid along with either pLVX-EF1alpha-SARS-CoV-2-nsp5-2xStrep-IRES-Puro (M^{pro} WT) (a gift from Nevan Krogan (Addgene plasmid #141370; http://n2t.net/addgene:141370; RRID:Addgene_141370) or pLVX-EF1alpha-SARS-CoV-2-nsp5-C145A-2xStrep-IRES-Puro (C145A mutant M^{pro}) plasmid (a gift from Nevan Krogan (Addgene plasmid #141371; http://n2t.net/addgene:141371; RRID:Addgene_141371). After 48 h of transfection, mGL fluorescence is measured using a Tecan SPARK® multimode microplate reader prior to bioluminescence measurements. Samples are excited at a wavelength 480 nm and emission is acquired at a wavelength of 530 nm. Bioluminescence is measured in the cells after addition of luciferase substrate, coelenterazine h, addition at a final concentration of 5 μM. Bioluminescence spectra are acquired using the Tecan SPARK® multimode microplate reader between 380 to 665 nm wavelengths with an acquisition time of 400 ms for each wavelength. Bioluminescence spectral data is normalized by dividing emissions at all wavelengths with that of emission at 483 nm. BRET is calculated as a ratio of emission at 516 nm (corresponding to the emission maxima of acceptor mGL) and 483 nm (corresponding to the emission maxima of donor picALuc). Bioluminescence of each sample is calculated from emissions at wavelengths between 380 and 665 nm and is first normalized with mGL fluorescence (relative to WT values) and then with that of bioluminescence of WT picALuc. All experiments are performed thrice in triplicates.

In vitro assays: In vitro assays are performed using lysates prepared from cells expressing the respective picALuc constructs as described previously. After 48 h of transfection, cells are washed thrice in chilled Dulbecco's phosphate-buffered saline (DPBS) and harvested and lysed by sonication on ice in a buffer containing 50 mM HEPES (pH 7.5), 100 mM NaCl, 2 mM EDTA, 1 mM dithiothreitol (DTT), 1×protease inhibitor cocktail (ThermoFisher Scientific, Massachusetts, USA) and 10% glycerol. Sonicated samples are centrifuged at 4° C. for 1 h and supernatant is collected for further experiments. For biochemical experiments to determine substrate affinity and reaction velocity, equivalent amounts of the proteins are taken after normalization with mGL fluorescence and incubated with a range of coelenterazine h concentrations. Data are fit to a allosteric sigmoidal model considering bioluminescence output (counts per second) representing the rate of reaction to determine Vₘₐₓ and K_{half} values. Thermal stability of the proteins is determined by measuring bioluminescence from equivalent amounts of cell lysates after incubation at a range of temperatures for 10 min. Data are fit to a Boltzmann sigmoidal model to determine the temperature at which the protein shows half maximal activity (Tₘ). Bioluminescence is measured after addition of the substrate in a GloMax® Discover Microplate Reader (Promega). All experiments are performed thrice in triplicates.

Data Analysis and Figure Preparation: MATLAB (release 2021b), Matplotlib, GraphPad Prism (version 9 for macOS, GraphPad Software, La Jolla California USA; www.graphpad.com) and Microsoft Excel are used for data analysis and graph preparation. Figures are assembled using Adobe Illustrator.

### Example 1: picALuc Structural Features and Reorganization

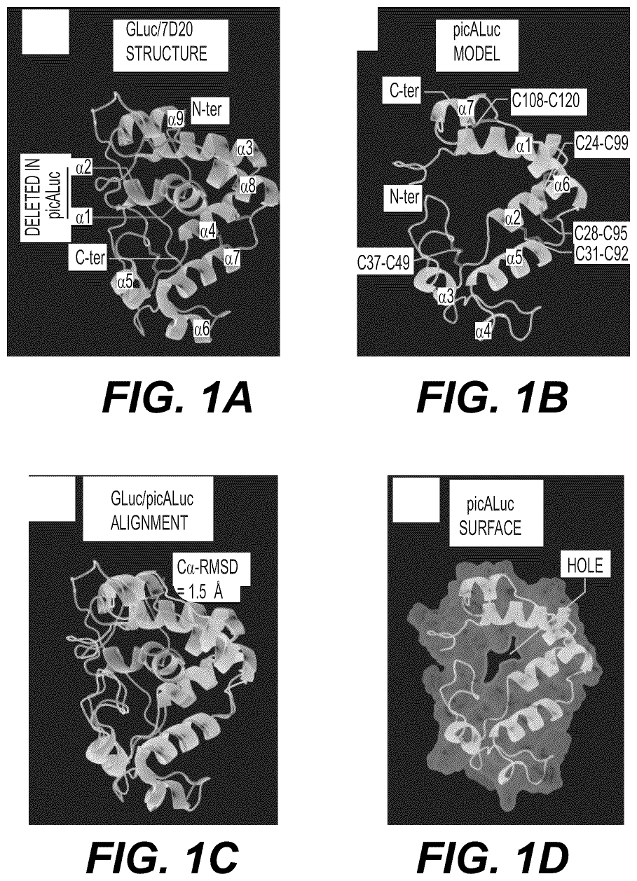

Deletion mutagenesis of ALuc, a synthetically designed luciferase protein, results in the generation of the smallest, 13 kDa, luciferase, picALuc. Specifically, deletion of residues 1 to 54 at the N-terminal (comprising of helices α1 and α2) and residues 175 to 194 at the C-terminal side (comprising of an extended unstructured loop region) is examined. To better understand the structural features of picALuc, we generate a structural model of the protein using the available NMR structure of GLuc. For this, the amino acid sequences of the picALuc and GLuc are aligned and a structural model is generated using the Modeller software (FIG. 1A-D). One of the key features of GLuc is the presence of five disulfide bridges formed by residue pairs C28-C95, C31-C92, C37-C49, C24-C99 and C108-C120 (FIG. 1A), which are maintained in the picALuc model. Additionally, all secondary structural elements are maintained in the modelled structure of picALuc (FIG. 1A, B), except for those which are deleted in picALuc (a helices in FIG. 1B are numbered according to the picALuc sequence). Overall, GLuc and the modelled picALuc structures show a Cα RMSD value of 1.5 Å (FIG. 1C). However, a closer inspection of the two structures reveals a non-compact structure with a ‘hole’ at the N-terminal side due to the absence of the helices α1 and α2, which are present in the GLuc structure but deleted in picALuc (FIG. 1D).

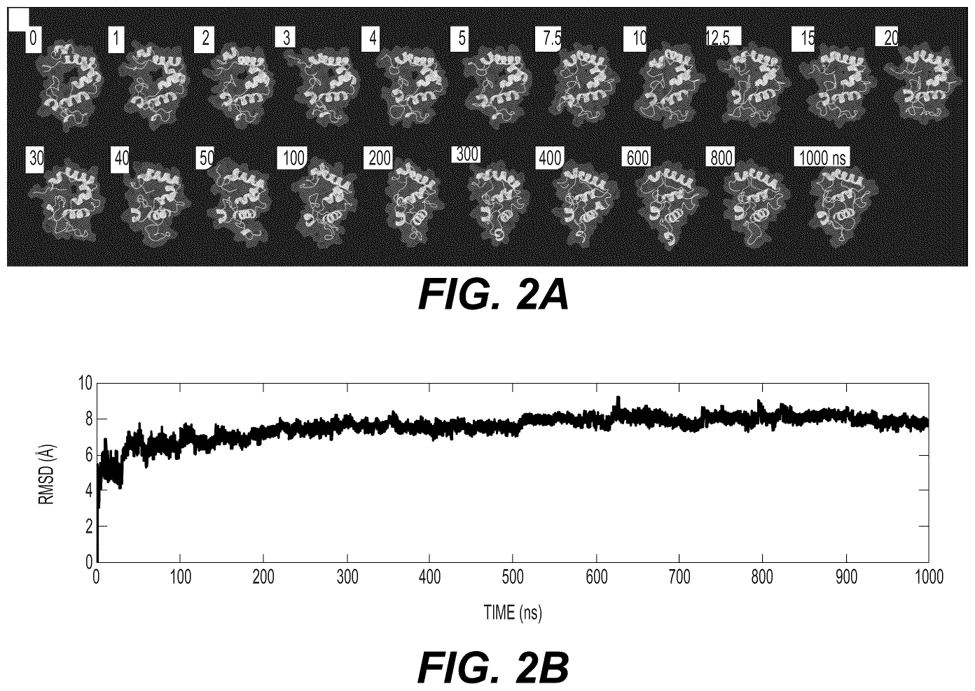

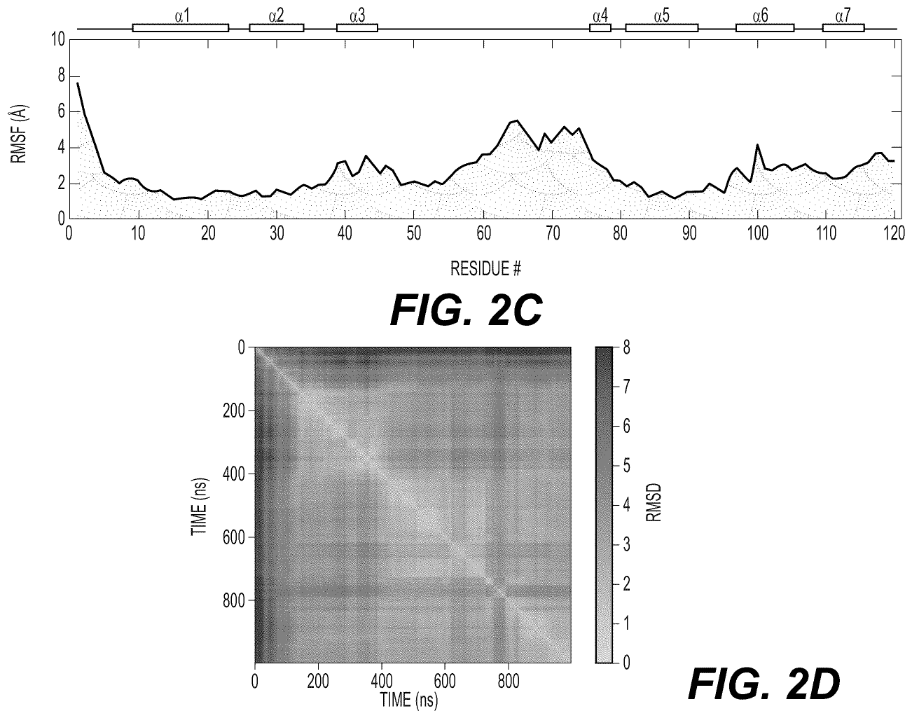

To determine if the modelled picALuc structure is stable or assumes a more compact form, we perform molecular dynamics (MD) simulations. For this, we set up an all atom, explicit solvent MD simulation and run the simulation for 1 μs using NAMD. We employ GaMD simulation to better explore the conformational dynamics of the protein within the simulation period of 1 μs. Analysis of the simulation trajectory reveals a rapid compaction of the picALuc structure within the first 10 ns of the simulation with further changes observed until 50 ns (FIG. 2A). Root mean square distance (RMSD) of Cα atoms reveals large changes in the initial phases of the simulation with stabilization of the RMSD trace observed after about 100 ns. Additionally, root mean square fluctuation (RMSF) analysis of the trajectory reveals largest fluctuations in residues corresponding to the loop between helices α3 and α4, residues corresponding to helix α3 in the N-terminal side and helices α6 and α7 in the C-terminal side (FIG. 2C). Further, pairwise RMSD analysis of the trajectory also captures the large structural changes in picALuc in the initial 100 ns of the simulation with largest changes seen in the first 10 ns of the simulation (FIG. 2D).

Solvent accessible surface area (SASA) and radius of gyration (RoG) analysis of the protein is performed to confirm the structural compaction. These reveal a general reduction in both SASA and RoG during the initial 200 ns of the simulation with somewhat slower decrease in the initial 100 ns but a much rapid decrease in between 100 and 200 ns. This is in contrast with the Cα RMSD and pairwise RMSD analyses which show large changes during the initial 100 ns and likely reflects side chain rearrangements and further packing in the protein. We also determine various types of energies of the protein and find that the van der Waal energy decreases in the initial phases of the simulation but reverts to similar level during the later phases of the simulation. Electrostatic (as well as non-bond and total) energy of the protein decreases during the initial phases of the simulation and remains low during the later phases of the simulation. However, the number of hydrogen bonds (H-bonds) also remains similar during the simulation. Additionally, secondary structure analysis reveals changes only in the C-terminal part of the loop between helices α3 and α4, indicating that the structural compaction of picALuc observed during the simulation is likely due to movement of the initially modelled a helices. We note that all five disulfide bridges are maintained all throughout the simulation.

### Example 2: Increased Number of Salt Bridge Interactions Formed in picALuc

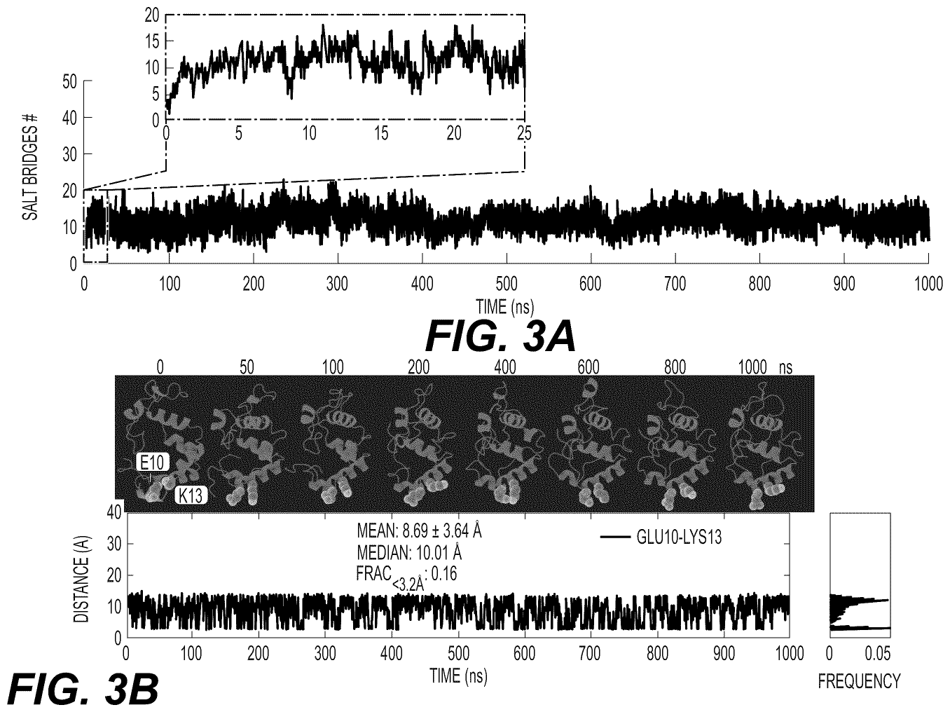

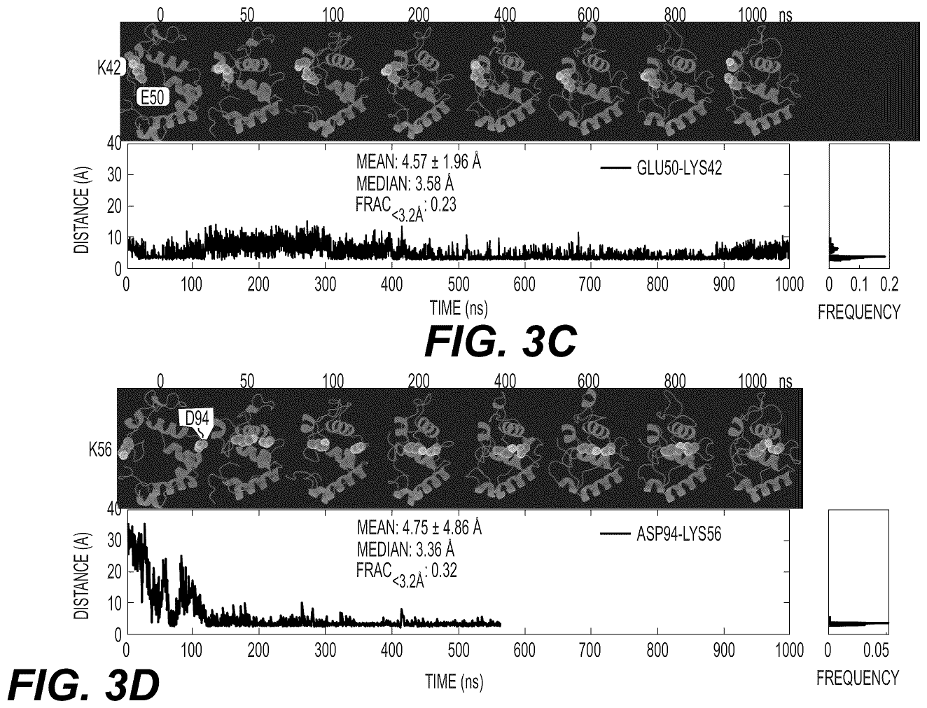

Given the observation of increased electrostatic interaction and the presence of a large number of charged residues in picALuc, we then focus our attention on the salt bridge interactions formed during the 1 μs long GaMD simulation. Determining the total number of salt bridge interactions formed with an oxygen-nitrogen distance cutoff of 3.2 Å during the simulation reveals an increase in the number of salt bridge interactions formed by the protein (FIG. 3A), with largest increases observed during the initial phase of the simulation. We, therefore, analyze the trajectory for salt bridge interactions formed during the simulation and find several salt bridge interactions that are found during the initial phase of the simulation while a number of such interactions are lost. In addition, several salt bridge interactions are found to be fluctuating or are formed transiently. For instance, residues E10 and K13 (both located in the helix α1) form a salt bridge interaction that shows fluctuating distance between the two amino acid residues with a mean (±s.d.) and median distance of 8.69±3.64 and 10.01 Å, respectively and a 16% fractional occupancy below 3.2 Å (FIG. 3B). We observe relatively stable salt bridge interactions formed between E16 and R26, E50 and K36 and E50 and K42 throughout the trajectory. For instance, residues E50 (located in the loop between helices α3 and α4) and K42 (located in the helix α3) form a relatively stable salt bridge interaction with a mean (±s.d.) and median distance of 4.57±1.96 and 3.58 Å, respectively and a 23% fractional occupancy below 3.2 Å (FIG. 3C). On the other hand, residues D94 (located in the loop between helices α5 and α6) and K56 (located in the loop between helices α3 and α4) form a new salt bridge interaction during the initial phase of the simulation with mean (±s.d.) and median distances of 4.75±4.86 and 3.36 Å, respectively and a 32% fractional occupancy below 3.2 Å (FIG. 3D).

### Example 3: Mutational Analysis Reveals Increased Bioluminescence of picALuc

Following our observations with the specific salt bridge interactions above, we next determine their role in the bioluminescence activity of picALuc. For this, we mutate the residues E10, E50 and D94 to A (E10A, E50A and D94A, respectively) and express the proteins, along with the wild type (WT) protein, in mammalian (HEK 293T) cells. Additionally, we fuse the mGreenLantern (mGL) green fluorescent protein at the N-terminal of picALuc for monitoring expression level of the proteins (FIG. 4A). Fluorescence measurement of living cells expressing the WT and the mutant picALuc proteins shows similar expression of the WT, E10A and E50A mutants while the D94A mutant shows higher levels of the protein (FIG. 4B). Bioluminescence measurement of the cells, on the other hand, shows similar activity of the WT and E10 Å and D94A mutant picALuc while the E50A mutant shows higher activity (FIG. 4C). Bioluminescence spectral measurements of the cells expressing the proteins show a single peak at around 526 nm (FIG. 4D) likely indicating a high efficiency of resonance energy transfer measured as a ratio of bioluminescence (FIG. 4E). Proteolytic cleavage of the SARS-CoV-2 M^{pro} N-terminal autocleavage site present in between mGL and picALuc upon co-expression of the M^{pro} results in a shift in the spectra and a significant decrease in the BRET (FIG. 9A, FIG. 9B). Overall, we observed that the E50A mutant picALuc shows about 3.8 times bioluminescence activity (after normalization with total protein levels as determined from mGL fluorescence) compared to the WT picALuc (FIG. 4F). Thus, while the residue E50 forms a stable salt bridge interaction (with the residue K42) throughout the 1 μs GaMD simulation, mutation of the same results in an increased bioluminescence activity of picALuc.

### Example 4: E50A Mutation Results an Increased Enzymatic Activity without Altering Thermal Stability of the Protein

Next, we determine the biochemical and thermal property of the protein in vitro. For this, HEK 293T cells transfected with the WT and various mutant picALuc proteins are used for preparing picALuc containing cell lysates to be used for in vitro assays. First, we perform enzyme kinetic studies with the WT and various mutants (equivalent concentrations of proteins are used) under a range of substrate (coelenterazine h) concentrations and utilize rate of photon emission as the enzymatic rate (bioluminescence; counts per second (CPS)) and fit the data to an allosteric sigmoidal model (FIG. 5A). Consistent with the results obtained with live cells, we observe a higher bioluminescence activity with the E50A mutant picALuc compared to the WT picALuc. While the rate constant (K_{half}) is found to be similar for all four proteins (FIG. 5B), the maximum enzyme velocity (Vₘₐₓ) is significantly higher for the E50A mutant compared to the WT picALuc (FIG. 5C). Additionally, we observe a significant decrease in the Vₘₐₓ of the E10A and D94A mutant compared to the WT picALuc (FIG. 5C), suggesting a role for these residues and likely the salt bridge interactions formed by these residues in the catalytic activity of picALuc.

Further, we determine the bioluminescence activity of the four picALuc proteins after incubation at a range of temperatures (from 32 to 84° C.) for 10 min. We fit the data to a 13oltzmann sigmoidal model to determine the melting temperature (Tₘ) of the proteins (FIG. 5D). We observe a relatively high thermal stability of picALuc with a Tₘ of 55.4±7.7° C. (FIG. 5E). However, no significant changes are observed in the Tₘ of the mutants, including E50A (FIG. 5E). This likely reflects the thermal stability provided by the five disulfide bridges in the protein. Taken together, these data suggest that the E50A mutation increases the bioluminescence activity of picALuc by increasing its Vₘₐₓ without affecting its thermal stability.

### Example 5: Altered Structural Dynamics in the E50A Mutant picALuc

To understand the mechanism for increased bioluminescence, we introduce the E50A mutation in the structural model of picALuc and perform an all atom, explicit solvent 1 μs GaMD simulation similar to the WT protein. We then analyze the trajectories of the WT and the E50A mutant picALuc to determine the distance between the Cα atoms in residues at position 50 (E in the WT and A in the E50A mutant picALuc) and 42 (K in both proteins) and find that the E50A mutation did not result in any major changes between the two proteins except for some differences in the initial phases of the simulation (FIG. 6A), likely reflecting the overall structural stability of the mutant protein. We then analyze the E50A mutant picALuc trajectory for Cα RMSF values and map the RMSF values of individual amino acid residues onto the structural conformer obtained from the last frame of the simulation as b-factors for the both the E50A mutant and WT picALuc. This reveals an increased fluctuation in the N-terminal residues and the long loop between helices α3 and α4 in the E50A mutant picALuc compared to the WT (FIG. 6B). In order to further understand the structural dynamics of the WT and E50A mutant picALuc, we utilize the dimensionality reduction analysis and perform principal component analysis (PCA) of their trajectories, which is known to reveal collective motions in proteins using the PCA module available in the Python-based MDAnalysis package. Cumulative variance analysis of the principal components reveals the presence of dominant collective motions in both the WT and the E50A mutant picALuc since principal components 1 and 2 (PC1 and PC2) can account for 69.8 and 19.2% of the variance in the WT protein while they can account for 74.2 and 19.8% of the variance in the E50A mutant protein. This analysis reveals large differences in the PC1 and PC2 of the two trajectories (FIG. 6C), suggesting changes in the collective dynamics of picALuc upon E50A mutation. We, therefore, determine the contribution of individual residues to the PC1 and 2 in the WT and the E50A mutant picALuc trajectory and find residues K42 and E50, other than the N-terminal residues and those from the loop α3-α4, contributing significantly to PC1 in the WT protein (FIG. 6D). Such contributions from both residue positions (K42 and A50) as well as those from helix α3 in general are lost in the PC1 of the E50A mutant picALuc (FIG. 6D). These data suggest a role for the salt bridge interaction formed by residues E50 and K42 in the collective dynamics of picALuc, and that this mode of collective motion likely restricts the catalytic activity of the protein.

MD simulation followed by detailed analysis of the trajectory not only reveals a compact three-dimensional structure of picALuc but also provides insights into the possible residue-level interactions. Specifically, closer inspection and mutational analysis of selected salt bridge forming residues enables the generation of a brighter picALuc variant. Comparative analysis of the WT and E50A mutant picALuc MD simulation trajectories reveal an altered collective motion and specifically the contribution of positions 42 and 50 in the mutant picALuc. The results may allow for the generation of new variants of the smallest luciferase proteins; highlight the role of collective dynamics in enzyme activity; and show that mutations in an enzyme can be selected based on their contribution in the collective dynamics of the protein.

Numerous references have been made to patents and printed publications throughout this specification. Each of the above-cited references and printed publications are individually incorporated herein by reference in their entirety.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

I claim:

1. A modified luciferase comprising a polypeptide having SEQ ID NO: 2 including at least one amino acid mutation, wherein the modified luciferase has a greater brightness than the polypeptide having SEQ ID NO: 2.

2. The modified luciferase of claim 1, wherein glutamine is replaced by alanine at amino acid 10, glutamine is replaced by alanine at amino acid 50, or aspartic acid is replaced by alanine at amino acid 94, or a combination thereof.

3. The modified luciferase of claim 1, wherein glutamine is replaced by alanine at amino acid 10.

4. The modified luciferase of claim 1, wherein glutamine is replaced by alanine at amino acid 50.

5. The modified luciferase of claim 1, wherein aspartic acid is replaced by alanine at amino acid 94.

6. A modified luciferase which exhibits greater brightness than luciferase of SEQ ID NO: 2, comprising a polypeptide having an amino acid sequence which differs from SEQ ID NO: 2 in that glutamine is replaced by alanine at amino acid 50.

7. The modified luciferase of claim 6, wherein the brightness exhibited is at least about two-fold greater than the brightness of luciferase of SEQ ID NO: 2.

8. The modified luciferase of claim 6, wherein the brightness exhibited is at about three-fold greater than the brightness of luciferase of SEQ ID NO: 2.

9. A polypeptide comprising an amino acid sequence selected from the group consisting of SEQ ID NO: 4, SEQ ID NO: 6, and SEQ ID NO: 8.

10. The polypeptide of claim 9, wherein the amino acid sequence is SEQ ID NO: 4.

11. The polypeptide of claim 9, wherein the amino acid sequence is SEQ ID NO: 6.

12. The polypeptide of claim 9, wherein the amino acid sequence is SEQ ID NO: 8.

13. A fusion protein comprising the polypeptide of claim 9.

14. A polynucleotide encoding the polypeptide of claim 9.

15. The polynucleotide of claim 14, wherein the amino acid sequence is SEQ ID NO: 4.

16. The polynucleotide of claim 14, wherein the amino acid sequence is SEQ ID NO: 6.

17. The polynucleotide of claim 14, wherein the amino acid sequence is SEQ ID NO: 8.

18. A vector comprising the polynucleotide of claim 14.

19. A host cell comprising the vector of claim 18.

20. A kit comprising the luciferase of claim 1.

21. A method of increasing the brightness of a luciferase comprising changing a salt bridge interaction of the luciferase.


SEQUENCE LISTING (12 sequences)

SEQ ID NO: 1 - DNA, 1299 bp, synthetic construct
  source 1..1299: mol_type = other DNA
atgggaagttcacatcatcatcatcatcactcatcaggactggtgccacgggggtctgaattcggcatggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttaggctacggcgtggcctgcttcgcccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgtgctgaagggcatcgacttcaaggaggacggcaacatcctggggcacaagctggagtacaacttcaacagccacaaggtctatatcacggccgacaagcagaagaacggcatcaaggctaacttcaagacccgccacaacgttgaggacggcggcgtgcagctcgccgaccactaccagcagaacacccccatcggcgacggccccgtgctgctgcccgacaaccactacctgagccatcagtccaaactgagcaaagaccccaacgagaagcgcgatcacatggtcctgaaggagagggtgaccgccgccgggattacacatgacatggacgagctgtacaagtacggatccgcggccgccaccgagaacctgtatgcagtgctccaaagcggatttcgcggctctggcagcgctatgaagctgcccggcaagaagctgcccctggaggtgctgaaggagctggaggccaacgcccagaaggccggctgcaccaggggctgcctgatctgcctgagccacatcaagtgcaccgccaagatgaagaagtggctgcccggcaggtgcgagagctgggagggcgacaaggagaccggccagggcggcatcggcgaggccatcgtggacatccccgagatccccggcttcaaggagctggcccccatggagcagttcatcgcccaggtggacctgtgcgccgactgcaccaccggctgcctgaagggcctggccaacgtgaagtgcagcgccctgctgaagaagtggctgcccagcaggtgcggtaccgactacaaagaccatgacggtgattataaagatcatgacatcgattacaaggatgacgatgacaaggatatctga

SEQ ID NO: 2 - protein, 432 aa, synthetic construct
MGSSHHHHHHSSGLVPRGSEFGMVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYKYGSAAATENLYAVLQSGFRGSGSAMKLPGKKLPLEVLKELEANAQKAGCTRGCLICLSHIKCTAKMKKWLPGRCESWEGDKETGQGGIGEAIVDIPEIPGFKELAPMEQFIAQVDLCADCTTGCLKGLANVKCSALLKKWLPSRCGTDYKDHDGDYKDHDIDYKDDDDKDI

SEQ ID NO: 3 - DNA, 1299 bp, synthetic construct
  source 1..1299: mol_type = other DNA
atgggaagttcacatcatcatcatcatcactcatcaggactggtgccacgggggtctgaattcggcatggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttaggctacggcgtggcctgcttcgcccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgtgctgaagggcatcgacttcaaggaggacggcaacatcctggggcacaagctggagtacaacttcaacagccacaaggtctatatcacggccgacaagcagaagaacggcatcaaggctaacttcaagacccgccacaacgttgaggacggcggcgtgcagctcgccgaccactaccagcagaacacccccatcggcgacggccccgtgctgctgcccgacaaccactacctgagccatcagtccaaactgagcaaagaccccaacgagaagcgcgatcacatggtcctgaaggagagggtgaccgccgccgggattacacatgacatggacgagctgtacaagtacggatccgcggccgccaccgagaacctgtatgcagtgctccaaagcggatttcgcggctctggcagcgctatgaagctgcccggcaagaagctgcccctggccgtgctgaaggagctggaggccaacgcccagaaggccggctgcaccaggggctgcctgatctgcctgagccacatcaagtgcaccgccaagatgaagaagtggctgcccggcaggtgcgagagctgggagggcgacaaggagaccggccagggcggcatcggcgaggccatcgtggacatccccgagatccccggcttcaaggagctggcccccatggagcagttcatcgcccaggtggacctgtgcgccgactgcaccaccggctgcctgaagggcctggccaacgtgaagtgcagcgccctgctgaagaagtggctgcccagcaggtgcggtaccgactacaaagaccatgacggtgattataaagatcatgacatcgattacaaggatgacgatgacaaggatatctga

SEQ ID NO: 4 - protein, 432 aa, synthetic construct
MGSSHHHHHHSSGLVPRGSEFGMVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYKYGSAAATENLYAVLQSGFRGSGSAMKLPGKKLPLAVLKELEANAQKAGCTRGCLICLSHIKCTAKMKKWLPGRCESWEGDKETGQGGIGEAIVDIPEIPGFKELAPMEQFIAQVDLCADCTTGCLKGLANVKCSALLKKWLPSRCGTDYKDHDGDYKDHDIDYKDDDDKDI

SEQ ID NO: 5 - DNA, 1299 bp, synthetic construct
  source 1..1299: mol_type = other DNA
atgggaagttcacatcatcatcatcatcactcatcaggactggtgccacgggggtctgaattcggcatggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttaggctacggcgtggcctgcttcgcccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgtgctgaagggcatcgacttcaaggaggacggcaacatcctggggcacaagctggagtacaacttcaacagccacaaggtctatatcacggccgacaagcagaagaacggcatcaaggctaacttcaagacccgccacaacgttgaggacggcggcgtgcagctcgccgaccactaccagcagaacacccccatcggcgacggccccgtgctgctgcccgacaaccactacctgagccatcagtccaaactgagcaaagaccccaacgagaagcgcgatcacatggtcctgaaggagagggtgaccgccgccgggattacacatgacatggacgagctgtacaagtacggatccgcggccgccaccgagaacctgtatgcagtgctccaaagcggatttcgcggctctggcagcgctatgaagctgcccggcaagaagctgcccctggaggtgctgaaggagctggaggccaacgcccagaaggccggctgcaccaggggctgcctgatctgcctgagccacatcaagtgcaccgccaagatgaagaagtggctgcccggcaggtgcgccagctgggagggcgacaaggagaccggccagggcggcatcggcgaggccatcgtggacatccccgagatccccggcttcaaggagctggcccccatggagcagttcatcgcccaggtggacctgtgcgccgactgcaccaccggctgcctgaagggcctggccaacgtgaagtgcagcgccctgctgaagaagtggctgcccagcaggtgcggtaccgactacaaagaccatgacggtgattataaagatcatgacatcgattacaaggatgacgatgacaaggatatctga

SEQ ID NO: 6 - protein, 432 aa, synthetic construct
MGSSHHHHHHSSGLVPRGSEFGMVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYKYGSAAATENLYAVLQSGFRGSGSAMKLPGKKLPLEVLKELEANAQKAGCTRGCLICLSHIKCTAKMKKWLPGRCASWEGDKETGQGGIGEAIVDIPEIPGFKELAPMEQFIAQVDLCADCTTGCLKGLANVKCSALLKKWLPSRCGTDYKDHDGDYKDHDIDYKDDDDKDI

SEQ ID NO: 7 - DNA, 1299 bp, synthetic construct
  source 1..1299: mol_type = other DNA
atgggaagttcacatcatcatcatcatcactcatcaggactggtgccacgggggtctgaattcggcatggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttaggctacggcgtggcctgcttcgcccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgtgctgaagggcatcgacttcaaggaggacggcaacatcctggggcacaagctggagtacaacttcaacagccacaaggtctatatcacggccgacaagcagaagaacggcatcaaggctaacttcaagacccgccacaacgttgaggacggcggcgtgcagctcgccgaccactaccagcagaacacccccatcggcgacggccccgtgctgctgcccgacaaccactacctgagccatcagtccaaactgagcaaagaccccaacgagaagcgcgatcacatggtcctgaaggagagggtgaccgccgccgggattacacatgacatggacgagctgtacaagtacggatccgcggccgccaccgagaacctgtatgcagtgctccaaagcggatttcgcggctctggcagcgctatgaagctgcccggcaagaagctgcccctggaggtgctgaaggagctggaggccaacgcccagaaggccggctgcaccaggggctgcctgatctgcctgagccacatcaagtgcaccgccaagatgaagaagtggctgcccggcaggtgcgagagctgggagggcgacaaggagaccggccagggcggcatcggcgaggccatcgtggacatccccgagatccccggcttcaaggagctggcccccatggagcagttcatcgcccaggtggacctgtgcgccgcctgcaccaccggctgcctgaagggcctggccaacgtgaagtgcagcgccctgctgaagaagtggctgcccagcaggtgcggtaccgactacaaagaccatgacggtgattataaagatcatgacatcgattacaaggatgacgatgacaaggatatctga

SEQ ID NO: 8 - protein, 432 aa, synthetic construct
MGSSHHHHHHSSGLVPRGSEFGMVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYKYGSAAATENLYAVLQSGFRGSGSAMKLPGKKLPLEVLKELEANAQKAGCTRGCLICLSHIKCTAKMKKWLPGRCESWEGDKETGQGGIGEAIVDIPEIPGFKELAPMEQFIAQVDLCAACTTGCLKGLANVKCSALLKKWLPSRCGTDYKDHDGDYKDHDIDYKDDDDKDI

SEQ ID NO: 9 - protein, 174 aa, synthetic construct
TGKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPAIPRFKDLEPMEQFIAQVDLCVDCTTGCLKGLANVQCSDLLKKWLPQRCATFASKIQGQVDKIKGAGGDIEGR

SEQ ID NO: 10 - protein, 114 aa, synthetic construct
KLPGKKLPLEVLKELEANAQKAGCTRGCLICLSHIKCTAKMKKWLPGRCESWEGDKETGQGGIGEAIVDIPEIPGFKELAPMEQFIAQVDLCADCTTGCLKGLANVKCSALLKK

SEQ ID NO: 11 - protein, 194 aa, synthetic construct
NHHHHHHHHDIVGVEGKFGTTDLETDLFTIVEDMNVISRDTDVDANRADRGRRGKLPGKKLPLEVLKELEANAQKAGCTRGCLICLSHIKCTAKMKKWLPGRCESWEGDKETGQGGIGEAIVDIPEIPGFKELAPMEQFIAQVDLCADCTTGCLKGLANVKCSALLKKWLPSRCAGFADKIQAQVDTIKGAGGS

SEQ ID NO: 12 - protein, 119 aa, synthetic construct
KLPGKKLPLEVLKELEANAQKAGCTRGCLICLSHIKCTAKMKKWLPGRCESWEGDKETGQGGIGEAVDIPEIPGFKELAPMEQFIAQVDLCADCTTGCLKGLANVKCSALLKKWLPSRC